(19) United States
(12) Patent Application Publication
(10) Pub. No.: US 2020/0094033 A1
(43) Pub. Date: Mar. 26, 2020

(54) MICRONEEDLE CURVED LAMINATE MOLD AND A METHOD OF MANUFACTURING MICRONEEDLE ARRAYS USING THIS MOLD

(71) Applicant: SABIC GLOBAL TECHNOLOGIES B.V., Bergen op Zoom (NL)

(72) Inventors: Johannes DE BROUWER, Oisterwijk (NL); Herwig JUSTER, Bergen Op Zoom (NL)

(21) Appl. No.: 16/471,957

(22) PCT Filed: Dec. 21, 2017
(86) PCT No.: PCT/IB2017/058317

§ 371 Date: Jun. 20, 2019

Related U.S. Application Data
(60) Provisional application No. 62/437,591, filed on Dec. 21, 2016.

(51) Int. Cl. A61M 37/00; B29C 43/22; B29C 33/42

(52) U.S. Cl. CPC A61M 37/0015; B29C 43/222; B29L 2031/7544; A61M 2037/0053; B29C 33/42

(57) ABSTRACT

A method of forming a microneedle array comprising disposing a first curved laminate 100 adjacent an embossing roll 101. The laminate comprises a body having a first longitudinal side and a second longitudinal side extending from a curved base. The first longitudinal side comprises a plurality of recesses 111 disposed adjacent a periphery of the first longitudinal side. At least a second curved laminate 105 is disposed adjacent the embossing roll, defining a plurality of cavities 208 there between. A material is extruded at one or more of the first and second curved laminates. At least a portion of the embossing roll is heated at a point of contact between the material and one or more of the first and second curved laminates. The material is caused to move into the plurality of cavities forming a plurality of projections at a surface of the material and the material is demolded.

### TECHNICAL FIELD

The application concerns forming microneedle arrays formed via variothermal extrusion and embossing techniques.

### BACKGROUND

Microneedles are attractive for delivery of certain therapeutics. These needles may be particularly desirable as a mode of therapeutic delivery because of the potential to replace syringe-with-needle type of injections with a pain free alternative. Microneedles can be virtually painless because they do not penetrate deep enough to contact nerves and only penetrate the outermost layer of the skin, unlike traditional syringes and hypodermic needles. Additionally, shallower penetration can also reduce the chance of infection or injury. Microneedles may also facilitate delivery of a more precise dosage of a therapeutic that enables the use of lower doses in treatments. Other advantages of microneedles for drug delivery include the simplified logistics (absence of required cold chain), ability for patient self-administration (no need for doctor, nurse, reduction of people transport). Beyond therapeutic delivery, drug delivery, microneedles have also been investigated for diagnostic applications. Bodily fluids coming out through the punctured skin can be analyzed for e.g. glucose or insulin.

Microneedles often require a manufacturing process that allows mass production at lowest cost, and as a consequence, shortest possible cycle time. In order to have proper transcription of mold texture and shape to the molded part, high flow may be necessary, especially having low viscosity at extremely high shear rates. Furthermore, good release from the production mold is important to reduce cycle time to improve the cost efficiency. These needles should have good strength to prevent breaking of the microneedle during usage. While there are a number of benefits to the use of microneedles and considerations with respect to forming them, certain challenges remain in microneedle production. It would be beneficial to prepare microneedles that exhibit a certain aspect ratio for a sharp tip and blade to puncture the skin.

### SUMMARY

Aspects of the invention concern methods of forming a microneedle array, the method comprising: disposing a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate cooperate with a second longitudinal side of the second curved laminate to define a plurality of cavities there between; extruding a material onto the one or more of the first or second curved laminate; heating at least a portion of the embossing roll via an external heat source at a point of contact between the material and the curved laminates; causing the material to move into the plurality of cavities to form a plurality of projections at a surface of the material, wherein the projections correspond to the plurality of cavities; and demolding the material from the curved laminates.

Other aspects may concern a system of forming a microneedle array, the system comprising: a first curved laminate adjacent an embossing roll, the curved laminate comprising a body having a first longitudinal side and a second longitudinal side extending from a curved base and wherein the first longitudinal side comprises a plurality of recesses disposed adjacent a periphery of the first longitudinal side; at least a second curved laminate disposed adjacent the first curved laminate such that the plurality of recesses of the first curved laminate cooperate with a second longitudinal side of the second curved laminate to define a plurality of cavities there between; a material configured to be contacted with the first or second curved laminate; a heat source configured to heat at least a portion of the first or second curved laminate; and a counter pressure roll disposed adjacent the first or second curved laminate, the counter pressure roll configured to apply a pressure at the replication material in contact with a heated portion of the first or second curved laminate to advance the material between the counter pressure roll and the first and second curved laminates thereby forming a plurality of projections at a surface of the replication material, wherein the plurality of projections correspond to a configuration for a microneedle array

### BRIEF DESCRIPTION OF THE DRAWINGS

FIG. 1 depicts a schematic diagram of curved laminate inserts at an embossing roll according to aspects of the present disclosure.

FIG. 2 depicts a schematic diagram of a curved laminate insert according to aspects of the present disclosure.

FIG. 3 depicts a cross-sectional view of curved laminates and recesses therein according to aspects of the present disclosure.

FIG. 4 depicts a schematic diagram of a cavity formed in the curved laminate mold according to aspects of the present disclosure.

FIGS. 5A-5C presents a diagram of exemplary types of embossing processes including roller embossing (5A), extrusion embossing (5B), and variothermal extrusion embossing (5C).

FIG. 6 presents a diagram for a variothermal extrusion process at a curved laminate mold according to aspects of the present disclosure.

### DETAILED DESCRIPTION OF ILLUSTRATIVE EMBODIMENTS

The present disclosure can be understood more readily by reference to the following detailed description of the disclosure and the Examples included therein.

Microneedles can be used to deliver a therapeutic or to draw blood without penetrating tissue as deep a traditional needles. Such microneedles can be used individually or as an array of needles. The needles are typically produced via mass production at a low cost. To function efficiently as a therapeutic delivery mechanism or as a diagnostic tool, microneedles must be sufficiently sharp to penetrate dermal surfaces while still maintaining the benefit of being relatively pain free. Thus, a given microneedle production array is desired to exhibit a certain aspect ratio among the formed microneedles while the formed needles still maintain their structural integrity and strength. The curved laminate mold and methods of forming a microneedle array therewith may provide a microneedle array having the desired varying aspect ratio sufficient to provide a sharp tip among the microneedles and a sharp blade to properly penetrate or cut the skin. A system for forming a microneedle array may comprise a plurality of curved laminates disposed adjacent an embossing roll, wherein the curved laminates comprise a plurality of half-pyramid cavities. A substrate may be deposited at a surface of the plurality of curved laminates and at least a portion of the substrate may be displaced into the plurality of cavities therein to form a microneedle array.

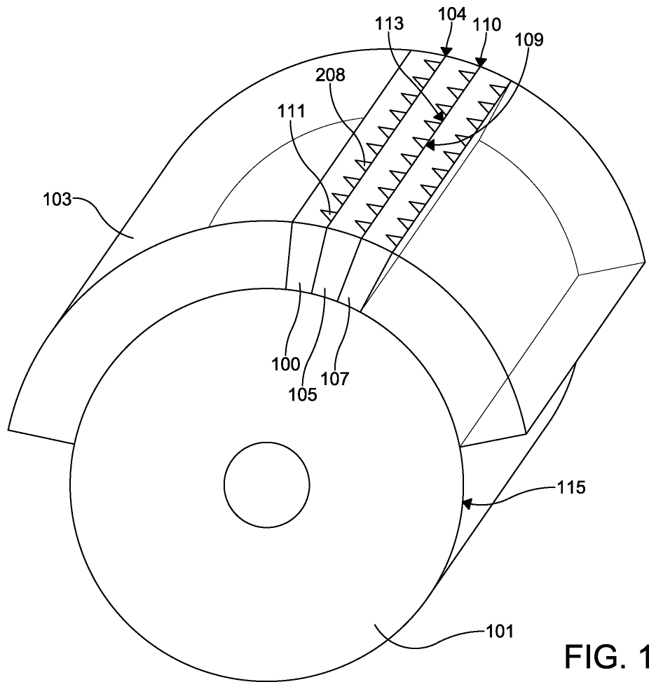

According to aspects of the present disclosure, a method of forming a microneedle array may comprise disposing a first curved laminate insert (or a first curved laminate) adjacent an embossing roll as shown in the cross sectional view of FIG. 1. In one aspect, a curved laminate mold for producing a microneedle array may be formed by disposing a first curved laminate 100 adjacent an embossing roll 101 in a laminate holder 103. One or more curved laminates disposed adjacent the embossing roll 101 may be referred to as a curved laminate mold. The laminate holder 103 may be configured to orient a plurality of curved laminate inserts adjacent to on another. The laminate holder 103 may be affixed to a surface 115 of the embossing roll 101. As an example, the laminate holder 103 may extend across at least a portion of a circumference of the embossing roll 101. In some aspects, the laminate holder 103 may abut, or be in contact with, the surface 115 of the embossing roll 101. In further aspects, the laminate holder 103 may be adjacent the embossing roll 101, but spaced therefrom.

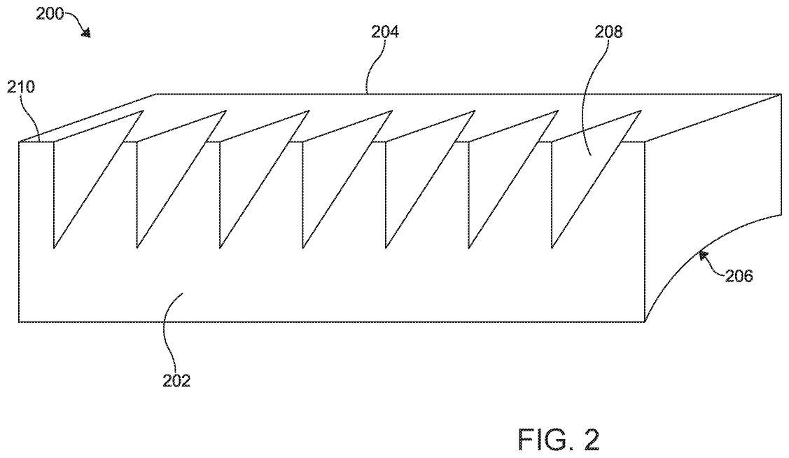

As shown in FIG. 2, a first curved laminate insert may comprise a body 200 having a first longitudinal side 202 and a second longitudinal side 204 extending from a curved base 206. The first longitudinal side 202 may comprise a plurality of recesses 208 disposed adjacent a periphery 210 of the first longitudinal side 202. Referring again to FIG. 1, to form a curved laminate mold, a second curved laminate insert 105 may be disposed adjacent the embossing roll 101 such that a plurality of recesses 108 of the first curved laminate 100 and a second longitudinal side 104 of the second curved laminate insert 105 cooperate to define a plurality of cavities 108 there between. That is, in one aspect, the plurality of recesses of the first curved laminate may be said to abut the second longitudinal side of the second curved laminate so that the second longitudinal side forms a wall, border, or enclosure for the plurality of recesses (described in FIG. 1).

In various aspects, a plurality of curved laminates may be used to form a curved laminate mold for a microneedle array formed by an embossing process. A plurality of curved laminates may be disposed adjacent an embossing roll configured for an embossing process. At least a third curved laminate insert 107 may be disposed adjacent the embossing roll 101 so that a second longitudinal side 109 of the third curved laminate insert 107 may define a second plurality of cavities 111 along an edge or periphery 110 of a first longitudinal side 113 of the second curved laminate 105.

The curved laminate mold may comprise a plurality of curved laminate inserts adjacent the embossing roll. As an example, the curved laminate mold may include up to about 21 curved laminate inserts disposed adjacent the embossing roll, forming up to about 20 sets of plurality of cavities there between. In an example, the curved laminate mold may include 11 curved laminates disposed adjacent the embossing roll.

A recess of the plurality of recesses within a curved laminate insert of the present disclosure may have a particular geometry. When a successive curved laminate is disposed adjacent a preceding curved laminate, a second longitudinal wall of the successive curved laminate may form a wall, border, or enclosure for a plurality of recesses along an edge or periphery of the first longitudinal side of the preceding curved laminate. For example, a second longitudinal side of a second curved laminate defines a right angle at each recess of the plurality of recesses disposed adjacent the periphery of the first longitudinal side, wherein the curved base is adjacent a surface of the embossing roll. That is, by orienting the curved laminates alongside, a backside for a recess of the plurality of recesses may be formed. The backside provides a 90° angle for enclosing the recess and forming a cavity. As a recess abuts or meets or contacts a side of an adjacent curved laminate insert, a cavity formed by the contact is defined. The cavity may exhibit a half-pyramid geometry. At least a portion of the cavities exhibit a half-pyramid geometry where two side lengths of the half-pyramid form an apex, corresponding to a penetrative point of a microneedle formed in the curved laminates mold. Each laminate cavity may thus have a certain base size and apex angle.

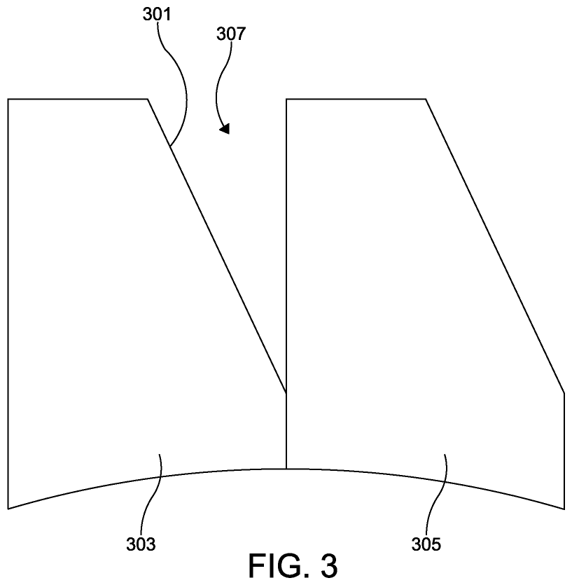

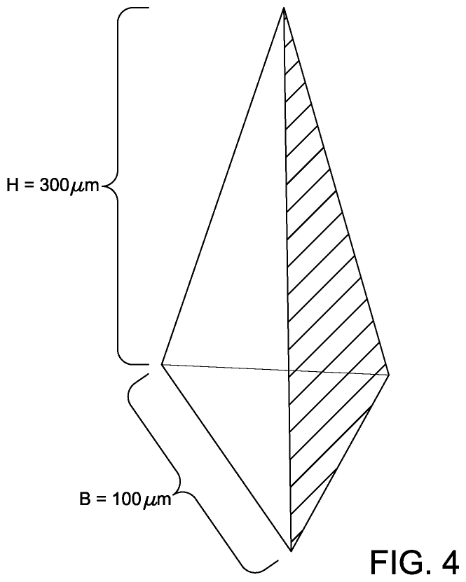

The half-pyramid cavity may have a depth of up to about 300 micrometers (μm), which may correspond to the height of a microneedle in a microneedle array formed according to the methods described herein. The plurality of recesses may be so spaced at a distance of about 1 millimeter from one another. FIG. 3 provides a cross-sectional view of a cavity 301 formed by a first curved laminate insert 303 and a second curved laminate insert 305 situated adjacent one another. A recess 301 of the first curved laminate may be said to abut a second longitudinal side 307 of the second curved laminate 305 so that the second longitudinal side 307 forms a wall, border, or enclosure at a right angle (or about 90°) with respect to a surface of the embossing roll for the recess 301, thereby defining the recess 301, now a cavity 301. FIG. 4 provides a schematic for geometry of a cavity according to the present disclosure. As shown, a base of the cavity may have a length of about 100 μm.

The present disclosure may combine a process of variothermal roller embossing process with a curved laminate mold disposed at a surface of an embossing roll to form a microneedle array. extruding a material onto the curved laminates; heating at least a portion of the embossing roll via an external heat source at a point of contact between the material and the curved laminates; causing the material to move into the plurality of cavities to form a plurality of projections at a surface of the material, wherein the projections correspond to the plurality of cavities; and demolding the material from the curved laminates.

A curved laminate mold may be formed by disposing a first curved laminate adjacent an embossing roll in a laminate holder. One or more curved laminates disposed adjacent the embossing roll may be referred to as a curved laminate mold. The curved laminate mold may be affixed to a surface of the embossing roller. The laminate holder may extend across a circumference of the embossing roll. The curved laminate mold may be comprised of up to about 21 curved laminates. As each curved laminate may comprise a plurality of recesses, the curved laminate mold may comprise up to 20 or up to about 20 repeating sets of the plurality of cavities as the curved laminates are situated alongside adjacent an embossing roll. A first recess of the plurality of recesses of a curved laminate may be spaced from another recess.

The curved laminate inserts described herein may be manufactured by a process of micro-electro discharge machining. The curved laminate inserts may be formed from a material such as stainless steel. As an example, the stainless

Forming a microneedle array may comprise a depositing a substrate at a curved laminate mold comprising a plurality of curved laminates adjacent an embossing roll. An appropriate embossing process may be employed to cause the substrate to be displaced into one or more cavities of a plurality of cavities disposed within the curved laminates. More specifically, forming a microneedle array may comprise disposing a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate cooperate with a second longitudinal side of the second curved laminate to define a plurality of cavities there between; extruding a substrate onto the one or more of the first or second curved laminate; heating at least a portion of the embossing roll via an external heat source at a point of contact between the material and the curved laminates; causing at least a portion of the substrate to be displaced into the plurality of cavities to form a plurality of projections at a surface of the material, wherein the projections correspond to the plurality of cavities; and demolding the material from the curved laminates.

Formation of a microneedle array at the curved laminate mold may be achieved by a number of molding processes configured to deposit a material into the plurality of cavities of the curved laminate mold. In various aspects, the curved laminate mold may be disposed at a roll of an embossing process. Embossing may be used to impart a texture or relief pattern into a number of products including textiles, paper, synthetic materials, metals, wood, and polymeric materials. In an embossing process, a substrate is caused to conform under pressure to the depths and contours of a pattern engraved or otherwise formed on an embossing roll. Embossing may be accomplished by passing a substrate through one or more patterned embossing rolls set to apply a certain pressure and penetration depth to the substrate. As the substrate traverses the embossing rolls, the pattern on the one or more rolls is imparted onto the substrate.

The patterns on embossing rolls may be mated or non-mated. In a pair of mated embossing rolls, the pattern on one of the rolls may identically, or similarly, compliment, or “mate,” with the pattern on a second or other of the mated rolls. The pattern on a non-mated embossing roll does not match identically with the pattern on the other roll. Depending on the desired results, either type of embossing roll can be used.

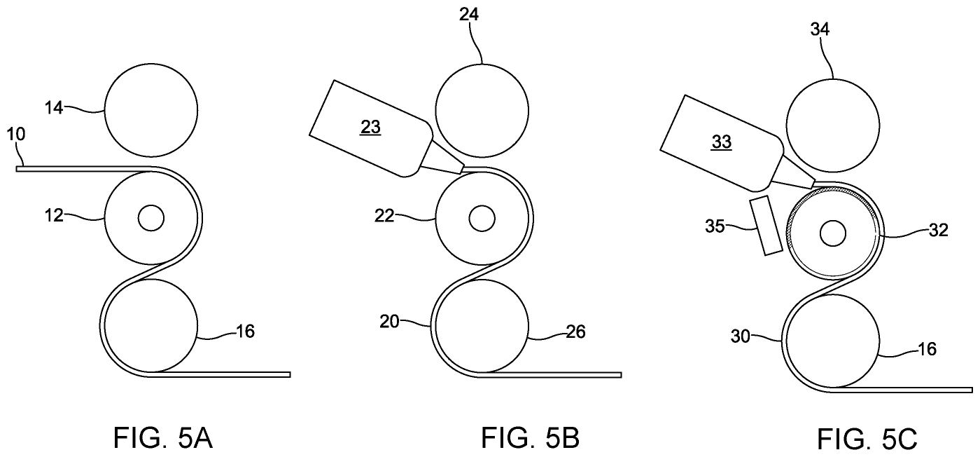

Various types of embossing processes may be useful in the formation of a microneedle array according to the methods described herein. These extrusion types may include, for example, roller embossing, extrusion embossing, and a variant on extrusion embossing referred to as variothermal extrusion embossing as shown in FIGS. 5A, 5B, and 5C, respectively. In a roller embossing process (FIG. 5A), a substrate (in the form of a sheet) 10 may be contacted with a rotating chill roll 12 having protrusions or depressions on a surface thereof. The chill roll 12 may be so named for its function of allowing the embossed substrate to cool and set into its embossed shape and may also be referred to as an embossing roll. A counter-pressure roll 14 may rotate to apply a force to depress the substrate 10 into depressions or protrusions on the surface of the chill roll 12 thereby delivering a reciprocal pattern onto the substrate 10. As described above, the chill roll 12 and the counter-pressure roll 14 may be “mated” or “non-mated.” A receiving roll 16 may receive a now embossed substrate 10 as it peels off the chill roll 12. The embossed substrate 10 from a roller embossing process may be thermally or ultraviolet UV radiation cured and the process enables a continuous production of polymeric films. Replication time (or formation of an embossed substrate) may be limited as a function of the rotation speed and diameter of the embossing roll. Pressure is generally not applied as the embossed substrate travels to the receiving roll in order to compensate against shrinkage. Demolding from the chill roll in roller embossing may occur with a peeling movement. Roller embossing may be advantageous for its production speed, which can reach 60 meters per minute (m/min) by a 2 meter width. The formation of deep structures as well as high-aspect ratios and replication quality however may present a challenge.

In extrusion embossing (FIG. 5B), a substrate 20 may be applied to a surface of the chill roll 22 via a direct feed from an extrusion die apparatus 23. Thus, the substrate 20 need only be used from material pellet form rather than forming a sheet prior to embossing. A rotating counter-pressure roll 24 may be used to apply a force at the extruded substrate 20 and a receiving roll 26 receives the substrate as it peels off from the embossing roll. For the variothermal variant of extrusion embossing (FIG. 5C), a substrate 30 may be applied to a surface of a chill roll 32 via a direct feed from an extrusion die apparatus 33. An external heating source 35 may be applied at an initial point of contact between the substrate 30 and the chill roll to heat the substrate 30 thereby facilitating formation of a reciprocal pattern that corresponds to depressions or protrusions at the heated surface of the chill roll 32. At least a portion of the chill roll 32 may be a cooling section. This cooling section may be disposed at a portion of the chill roll 32 which opposes at least a portion of the area of the chill roll 32 that has been heated by the external heating source (see grayscale at chill roll 32). A rotating counter-pressure 34 roll may be used to apply a force at the extruded, heated substrate 30 while a receiving roll 36 receives the now embossed substrate 30 as it cools and peels away from the chill roll 32.

According to various aspects of the present disclosure, variothermal embossing may be combined with the use of a curved laminate mold described herein may improve production of a microneedle array. Thermal management at a polymer substrate forming the microneedle array may provide a faster production rate because of the increased viscosity of the polymer substrate material while the laser drilled band mold at a chill roll may improve the quality of replication during embossing. The curved laminate mold may be disposed adjacent an embossing roll to form a chill roll in a variothermal embossing process. That is, the band mold may be fitted about an embossing roll and configured to receive a substrate. The chill roll may be configured to facilitate thermal management of the curved laminate mold. The method of the present disclosure may combine variothermal embossing with a single-step extrusion roller embossing process to provide a microneedle array. The chill roll (including the curved laminates mold) may be used as a mold for the microneedle array; the plurality of cavities within the curved laminate inserts forming the curved laminate mold may exhibit an inverse geometry suitable for microneedles. Variothermal heating may be used to obtain a better heat and cooling distribution on the chill roll thereby facilitating better microneedle replication. Specifically, the external heating source may generate a temperature profile along a circumference of the chill roll which is cooled.

The curved laminates mold may be disposed adjacent, about, around, or on a chill roll in a variothermal extrusion process to form a microneedle array. Forming the microneedle array may comprise disposing a curved laminates mold adjacent an embossing roll, the curved laminate mold having a plurality of curved laminate inserts therein, each curved laminate having a plurality of cavities therein. The embossing roll may be configured to cool at least a portion of the curved laminates mold. A material or substrate may be deposited on to the curved laminates mold and heat may be applied at a point of contact between the substrate and the curved laminates mold. The substrate may then be caused to move into the plurality of cavities of the curved laminates mold, thereby forming one or more projections at a surface of the substrate wherein the projections correspond to the cavities of the curved laminates mold. The substrate may be demolded from the surface of the curved laminates mold to form a microneedle array.

In some aspects, the conical depressions formed in the band mold via laser ablation may be oriented in a specific repeating pattern. In further examples however, the conical depressions may be randomly distributed at the band mold. The orientation of conical depressions in the band mold may thus correspond to a pattern in the resulting microneedle array or may provide a microneedle array in a random configuration.

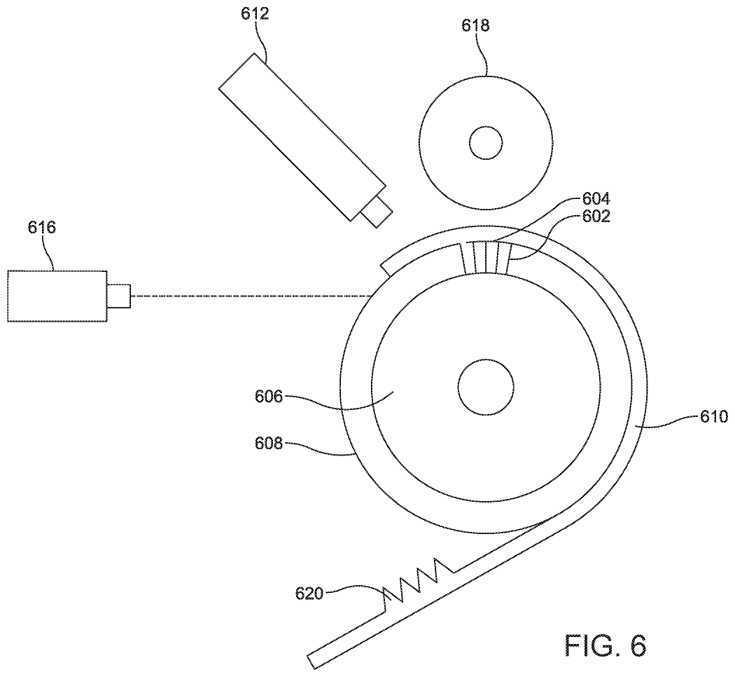

As shown in FIG. 6, to form a microneedle array, curved laminate inserts 602 (comprising a plurality of cavities 604) may be disposed adjacent an embossing roll 606 to form a chill roll 608. A substrate 610 may be deposited via an extrusion die apparatus 612 at a surface of the curved laminate inserts 602 comprising the plurality of cavities 604. While the curved laminate inserts 602 are disposed adjacent the embossing roll 606, heat may be applied via an external heat source 616 causing the substrate 610 to deform and be displaced into the plurality of cavities 604 of the curved laminate inserts 602. Heat may be applied to the curved laminate inserts 602 at or adjacent a point of contact between the substrate 610 and the curved laminate inserts 602. The chill roll 608 may thus operate as a heat sink during the application of heat. A counter-pressure roll 618 disposed adjacent the curved laminae inserts 602 may rotate in a direction opposing a rotation of the chill roll 608 thereby advancing the substrate 610 between the curved laminate inserts and the counter-pressure roll 618 and continuing to displacing at least a portion of the substrate 610 into the plurality of cavities 604. Displacement of the substrate 610 may form one or more projections 620 at the substrate 610 such that the one or more projections 620 correspond to the plurality of cavities 604 at the curved laminate inserts 602. The substrate 610 may demold or haul off from the chill roll 608 to provide a microneedle array.

Heating of at least a portion of the curved laminate inserts may comprise heating at least a portion of the band mold to a temperature above the melting point of the substrate. While at least one portion of the band mold is heated to cause deformation of the substrate into the plurality of cavities of the curved laminate inserts, at least a second portion of the curved laminate inserts may be maintained at a temperature less than the melting point of the substrate. The chill roll may be configured to be cooled in order to maintain a temperature less than the melting temperature of the substrate. In certain examples, demolding the substrate from the curved laminate inserts may comprise cooling at least a portion of the curved laminate inserts via cooling of the chill roll.

Various types of heating sources may be appropriate for the present disclosure. In some examples, the heating source is an external heating device comprising a diode laser system. The diode laser system may have a wavelength between 940 nanometers (nm) and 980 nm and may have a heating zone of at least about 10 millimeters (mm) in length and at least about 68 mm in width. An external heating source may be disposed adjacent the embossing roll/chill roll. The external heating source may be disposed within at least about 300 mm from the embossing roll/chill roll. In one example, a laser diode system heating device may apply heat at an angle of irradiation of 18° about 18°.

A process of forming a microneedle array as described herein comprising depositing a substrate at a curved laminate mold adjacent an embossing roll, the curved laminate mold comprising a plurality of curved laminate inserts having a plurality of cavities therein. An appropriate embossing process may then be employed to cause the substrate to be displaced into one or more cavities of a plurality of cavities disposed within the curved laminates. The disclosed process may facilitate mass production of microneedle arrays with varying needle geometries on a single roll type. Different microneedle geometries may be obtained by varying a base size of the plurality of cavities of the curved laminate inserts. Moreover, the process may enable continuous production of a film (via extrusion of the desired substrate) and subsequent embossing of microneedles on a single roll.

In various aspects, the substrate may comprise a polymer material. The substrate for forming a microneedle array in the disclosed variothermal embossing process may comprise a polymer or a mixture of polymers. Generally, the polymer mixture may be supplied in a liquid or flowable state, via for example, an extrusion die apparatus, to the band mold. The solid product comprising the microneedle array may then separate from the band mold. Exemplary polymer materials may comprise engineering thermoplastics such as polycarbonates, polyetherimides, polyphenylene ether, and polybutylene terephthalate, as well as blends of polycarbonate with acrylic butadiene styrene plastics.

The polymer material for forming the microneedle array may further comprise one or more additives intended to impart certain characteristics to a microneedle array formed by the mold assembly described herein. The polymer material may include one or more of an impact modifier, flow modifier, antioxidant, heat stabilizer, light stabilizer, ultraviolet (UV) light stabilizer, UV absorbing additive, plasticizer, lubricant, antistatic agent, anti-fog agent, antimicrobial agent, colorant (e.g., a dye or pigment), surface effect additive, radiation stabilizer, anti-drip agent (e.g., a polytetrafluoroethylene (PTFE)-encapsulated styrene-acrylonitrile copolymer (TSAN)), or a combination comprising one or more of the foregoing. For example, a combination of a heat stabilizer, and ultraviolet light stabilizer can be used. In general, the additives are used in the amounts generally known to be effective. For example, the total amount of the additive composition can be 0.001 weight percent (wt %) to 10.0 wt %, or 0.01 wt % to 5 wt %, each based on the total weight of all ingredients in the composition.

The polymer material may include various additives ordinarily incorporated into polymer compositions, with the proviso that the additive(s) are selected so as to not significantly adversely affect the desired properties of the thermoplastic composition (good compatibility for example). Such additives can be mixed at a suitable time during the mixing of the components for forming the composition.

In addition, the polymer material may exhibit excellent release, as measured by ejection force (N) and coefficient of friction. The polymer material also preferably show (i) high flow at high shear conditions to allow good transcription of mold texture and excellent filling of the finest mold features, (ii) good strength and impact, and (iii) high release to have efficient de-molding and reduced cooling and cycle time during molding. The microneedles formed herein may have sufficient mechanical strength to remain intact (i) while being inserted into the biological barrier, (ii) while remaining in place for up to a number of days, and (iii) while being removed.

### Aspects

The present disclosure comprises at least the following aspects.

Aspect 1A. A method of forming a microneedle array, the method comprising: disposing a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate cooperate with a second longitudinal side of the second curved laminate to define a plurality of cavities there between; extruding a material onto the one or more of the first or second curved laminate; heating at least a portion of the embossing roll via an external heat source at a point of contact between the material and the curved laminates; causing the material to move into the plurality of cavities to form a plurality of projections at a surface of the material, wherein the projections correspond to the plurality of cavities; and demolding the material from the curved laminates.

Aspect 1B. A method of forming a microneedle array, the method consisting essentially of: disposing a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate cooperate with a second longitudinal side of the second curved laminate to define a plurality of cavities there between; extruding a material onto the one or more of the first or second curved laminate; heating at least a portion of the embossing roll via an external heat source at a point of contact between the material and the curved laminates; causing the material to move into the plurality of cavities to form a plurality of projections at a surface of the material, wherein the projections correspond to the plurality of cavities; and demolding the material from the curved laminates.

Aspect 1C. A method of forming a microneedle array, the method consisting of: disposing a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate cooperate with a second longitudinal side of the second curved laminate to define a plurality of cavities there between; extruding a material onto the one or more of the first or second curved laminate; heating at least a portion of the embossing roll via an external heat source at a point of contact between the material and the curved laminates; causing the material to move into the plurality of cavities to form a plurality of projections at a surface of the material, wherein the projections correspond to the plurality of cavities; and demolding the material from the curved laminates.

Aspect 2. The method of aspect 1, further comprising disposing at least a third curved laminate adjacent the embossing roll so that a second longitudinal side of the third curved laminate defines a plurality of cavities along a periphery of the second curved laminate.

Aspect 3. The method of any one of aspects 1-2, wherein of the plurality of recesses exhibit a half pyramid geometry.

Aspect 4. The method of any one of aspects 1-3, further comprising a total of up to about 21 curved laminates.

Aspect 5. The method of any one of aspects 1-4, further comprising disposing up to an eleventh curved laminate at the embossing roll.

Aspect 6. The method of any one of aspects 1-5, wherein the second longitudinal side of the second curved laminate defines a right angle at each recess of the plurality of recesses disposed adjacent the periphery of the first longitudinal side, wherein the curved base is adjacent a surface of the embossing roll.

Aspect 7. The method of any one of aspects 1-6, wherein the recesses are spaced from each other.

Aspect 8. The method of any one of aspects 1-7, wherein the first curved laminate is disposed at the embossing roll in a laminate holder, wherein the laminate holder extends across a diameter of the embossing roll.

Aspect 9. The method of any one of aspects 1-8, wherein disposing the first curved laminate at the embossing roll comprises affixing the first curved laminate to the embossing roll.

Aspect 10. The method of any one of aspects 1-9, wherein causing the material to move into the plurality of cavities comprises engaging a counter pressure roll disposed adjacent the embossing roll to apply a pressure at the material at a portion of the material in contact with a heated portion of the embossing roll thereby advancing the material between the counter pressure roll and the embossing roll.

Aspect 11. The method of any one of aspects 1-10, wherein the plurality of cavities comprises ten cavities.

Aspect 12. The method of any one of aspects 1-11, wherein the curved laminates comprise steel.

Aspect 13. The method of any one of aspects 1-12, wherein the material comprises a homogenous thermoplastic melt.

Aspect 14. The method of any one of aspects 1-13, wherein the at least a portion of the embossing roll is heated to a temperature above a melting temperature of the material.

Aspect 15. The method of any one of aspects 1-14, wherein demolding the material from the curved laminates provides the material having the plurality of projections in a configuration corresponding to a microneedle array.

Aspect 16. The method of any one of aspects 3-15, wherein a side length of the half-pyramid geometry of the recesses is 300 micrometer and a base length of the half pyramid geometry of the recesses is 100 micrometer.

Aspect 17. The method of any one of aspects 3-15, wherein the half-pyramid geometry of the recesses corresponds to an aspect ratio between about 1:2 to about 1:4 based on a side length and a base length of the half-pyramid geometry.

Aspect 18A. A microneedle array formed by a process comprising: disposing at least a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate and a second longitudinal side of the second curved laminate cooperate to define a plurality of cavities there between; extruding a material onto the at least first and second curved laminates; heating at least a portion of the embossing roll via an external heat source at a point of contact between the material and the at least first and second curved laminates; causing the material to move into the plurality of cavities to form a plurality of projections at a surface of the material, wherein the projections correspond to the plurality of cavities; and demolding the material from the at least first and second curved laminates.

Aspect 18B. A microneedle array formed by a process consisting essentially of: disposing at least a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate and a second longitudinal side of the second curved laminate cooperate to define a plurality of cavities there between; extruding a material onto the at least first and second curved laminates; heating at least a portion of the embossing roll via an external heat source at a point of contact between the material and the at least first and second curved laminates; causing the material to move into the plurality of cavities to form a plurality of projections at a surface of the material, wherein the projections correspond to the plurality of cavities; and demolding the material from the at least first and second curved laminates.

Aspect 18C. A microneedle array formed by a process consisting of: disposing at least a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side; disposing at least a second curved laminate adjacent the embossing roll such that the plurality of recesses of the first curved laminate and a second longitudinal side of the second curved laminate cooperate to define a plurality of cavities there between; extruding a material onto the at least first and second curved laminates; heating at least a portion of the embossing roll via an external heat source at a point of contact between the material and the at least first and second curved laminates; causing the material to move into the plurality of cavities to form a plurality of projections at a surface of the material, wherein the projections correspond to the plurality of cavities; and demolding the material from the at least first and second curved laminates.

Aspect 19. The microneedle array of any of aspects 18A-18C, wherein the plurality of cavities have a half-pyramid geometry.

Aspect 20A. A system of forming a microneedle array, the system comprising: a first curved laminate adjacent an embossing roll, the curved laminate comprising a body having a first longitudinal side and a second longitudinal side extending from a curved base and wherein the first longitudinal side comprises a plurality of recesses disposed adjacent a periphery of the first longitudinal side; and at least a second curved laminate disposed adjacent the first curved laminate such that the plurality of recesses of the first curved laminate cooperate with a second longitudinal side of the second curved laminate to define a plurality of cavities there between; a heat source configured to heat at least a portion of the first or second curved laminate; and a counter pressure roll disposed adjacent the first or second curved laminate, the counter pressure roll configured to apply a pressure at a material in contact with a heated portion of the first or second curved laminate to advance the material between the counter pressure roll and the first and second curved laminates thereby forming a plurality of projections at a surface of the replication material, wherein the plurality of projections correspond to a configuration for a microneedle array.

Aspect 20B. A system of forming a microneedle array, the system consisting essentially of: a first curved laminate adjacent an embossing roll, the curved laminate comprising a body having a first longitudinal side and a second longitudinal side extending from a curved base and wherein the first longitudinal side comprises a plurality of recesses disposed adjacent a periphery of the first longitudinal side; and at least a second curved laminate disposed adjacent the first curved laminate such that the plurality of recesses of the first curved laminate cooperate with a second longitudinal side of the second curved laminate to define a plurality of cavities there between; a heat source configured to heat at least a portion of the first or second curved laminate; and a counter pressure roll disposed adjacent the first or second curved laminate, the counter pressure roll configured to apply a pressure at a material in contact with a heated portion of the first or second curved laminate to advance the material between the counter pressure roll and the first and second curved laminates thereby forming a plurality of projections at a surface of the replication material, wherein the plurality of projections correspond to a configuration for a microneedle array.

Aspect 20C. A system of forming a microneedle array, the system consisting of: a first curved laminate adjacent an embossing roll, the curved laminate comprising a body having a first longitudinal side and a second longitudinal side extending from a curved base and wherein the first longitudinal side comprises a plurality of recesses disposed adjacent a periphery of the first longitudinal side; and at least a second curved laminate disposed adjacent the first curved laminate such that the plurality of recesses of the first curved laminate cooperate with a second longitudinal side of the second curved laminate to define a plurality of cavities there between; a heat source configured to heat at least a portion of the first or second curved laminate; and a counter pressure roll disposed adjacent the first or second curved laminate, the counter pressure roll configured to apply a pressure at a material in contact with a heated portion of the first or second curved laminate to advance the material between the counter pressure roll and the first and second curved laminates thereby forming a plurality of projections at a surface of the replication material, wherein the plurality of projections correspond to a configuration for a microneedle array.

Aspect 21. The system of any of aspects 20A-20C, further comprising a plurality of successive curved laminates forming a plurality of successive cavities configured to receive the material.

Aspect 22A. A system of forming a microneedle array, the system comprising: a first curved laminate adjacent an embossing roll, the curved laminate comprising a body having a first longitudinal side and a second longitudinal side extending from a curved base and wherein the first longitudinal side comprises a plurality of recesses disposed adjacent a periphery of the first longitudinal side; and at least a second curved laminate disposed adjacent the first curved laminate such that the plurality of recesses of the first curved laminate cooperate with a second longitudinal side of the second curved laminate to define a plurality of cavities there between.

Aspect 22B. A system of forming a microneedle array, the system consisting essentially of: a first curved laminate adjacent an embossing roll, the curved laminate comprising a body having a first longitudinal side and a second longitudinal side extending from a curved base and wherein the first longitudinal side comprises a plurality of recesses disposed adjacent a periphery of the first longitudinal side; and at least a second curved laminate disposed adjacent the first curved laminate such that the plurality of recesses of the first curved laminate cooperate with a second longitudinal side of the second curved laminate to define a plurality of cavities there between.

Aspect 22C. A system of forming a microneedle array, the system consisting of: a first curved laminate adjacent an embossing roll, the curved laminate comprising a body having a first longitudinal side and a second longitudinal side extending from a curved base and wherein the first longitudinal side comprises a plurality of recesses disposed adjacent a periphery of the first longitudinal side; and at least a second curved laminate disposed adjacent the first curved laminate such that the plurality of recesses of the first curved laminate cooperate with a second longitudinal side of the second curved laminate to define a plurality of cavities there between.

Aspect 23. The system of any of aspects 22A-22C, further comprising a heat source configured to heat at least a portion of the first or second curved laminate; and a counter pressure roll disposed adjacent the first or second curved laminate, the counter pressure roll configured to apply a pressure at a material in contact with a heated portion of the first or second curved laminate to advance the material between the counter pressure roll and the first and second curved laminates thereby forming a plurality of projections at a surface of the replication material, wherein the plurality of projections correspond to a configuration for a microneedle array.

Aspect 24. The system of any one of aspects 22A-23, wherein causing the material to move into the plurality of cavities comprises engaging a counter pressure roll disposed adjacent the embossing roll to apply a pressure at the material at a portion of the material in contact with a heated portion of the embossing roll thereby advancing the material between the counter pressure roll and the embossing roll.

Aspect 25. The system of any one of aspects 22-24, wherein the plurality of cavities comprises ten cavities.

Aspect 26. The system of any one of aspects 22-25, wherein the curved laminates comprise steel.

Aspect 27. The method of any one of aspects 22-26, wherein the at least a portion of the embossing roll is heated to a temperature above a melting temperature of the material.

Aspect 28. The method of any one of aspects 22-27, wherein demolding the material from the curved laminates provides the material having the plurality of projections in a configuration corresponding to a microneedle array.

Aspect 29. The method of any one of aspects 22-28, wherein a side length of the half-pyramid geometry of the recesses is 300 micrometer and a base length of the half pyramid geometry of the recesses is 100 micrometer.

Aspect 30A. A mold for of forming a microneedle array, the mold comprising: a first curved laminate adjacent an embossing roll, the curved laminate comprising a from a first curved base and first body comprises a plurality of recesses disposed adjacent a periphery of the first body; and at least a second curved laminate having a second body extending from a second curved base, the second curved laminate disposed adjacent the first curved laminate such that the plurality of recesses of the first curved laminate cooperate with the second body of the second curved laminate to define a plurality of cavities there between.

Aspect 30B. A mold for of forming a microneedle array, the mold consisting essentially of: a first curved laminate adjacent an embossing roll, the curved laminate comprising a from a first curved base and first body comprises a plurality of recesses disposed adjacent a periphery of the first body; and at least a second curved laminate having a second body extending from a second curved base, the second curved laminate disposed adjacent the first curved laminate such that the plurality of recesses of the first curved laminate cooperate with the second body of the second curved laminate to define a plurality of cavities there between.

Aspect 30C. A mold for of forming a microneedle array, the mold consisting of: a first curved laminate adjacent an embossing roll, the curved laminate comprising a from a first curved base and first body comprises a plurality of recesses disposed adjacent a periphery of the first body; and at least a second curved laminate having a second body extending from a second curved base, the second curved laminate disposed adjacent the first curved laminate such that the plurality of recesses of the first curved laminate cooperate with the second body of the second curved laminate to define a plurality of cavities there between.

Aspect 31. The mold of any of aspects 30A-30C, wherein the mold is disposed adjacent an embossing roll such that the first curved base and second curved base are adjacent a surface of the embossing roll.

### Definitions

It is to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein the terms “weight percent,” “weight %,” and “wt. %” of a component, which can be used interchangeably, unless specifically stated to the contrary, are based on the total weight of the formulation or composition in which the component is included. For example if a particular element or component in a composition or article is said to have 8% by weight, it is understood that this percentage is relative to a total compositional percentage of 100% by weight.

As used herein, the terms “weight average molecular weight” or “Mw” can be used interchangeably, and are defined by the formula:

$${M}_{w} = \frac{\sum {N}_{i} {M}_{i}^{2}}{\sum {N}_{i} {M}_{i}} ,$$

where Mi is the molecular weight of a chain and Ni is the number of chains of that molecular weight. Mw can be determined for polymers, e.g. polycarbonate polymers, by methods well known to a person having ordinary skill in the art using molecular weight standards, e.g. polycarbonate standards or polystyrene standards, preferably certified or traceable molecular weight standards. Polystyrene basis refers to measurements using a polystyrene standard.

The term “siloxane” refers to a segment having a Si-O-Si linkage.

The term “flowable” means capable of flowing or being flowed. Typically a polymer is heated such that it is in a melted state to become flowable. ° C. is degrees Celsius. μm is micrometer.

1 It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of forming a microneedle array, the method comprising:
disposing a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side;
disposing at least a second curved laminate adjacent the embossing roll such that the plurality of recesses of the first curved laminate cooperate with a second longitudinal side of the second curved laminate to define a plurality of cavities there between;
extruding a material onto the one or more of the first and second curved laminate;
heating at least a portion of the embossing roll via an external heat source at a point of contact between the material and one or more of the first and second curved laminates;
causing the material to move into the plurality of cavities to form a plurality of projections at a surface of the material, wherein the projections correspond to the plurality of cavities; and
demolding the material from one or more of the first or second curved laminates.

2. The method of claim 1, further comprising disposing at least a third curved laminate adjacent the embossing roll so that a second longitudinal side of the third curved laminate defines a plurality of cavities along a periphery of the second curved laminate.

3. The method of claim 1, wherein of the plurality of recesses exhibit a half pyramid geometry.

4. The method of claim 1, further comprising a total of up to about 21 curved laminates.

5. The method of claim 1, further comprising disposing up to an eleventh curved laminate at the embossing roll.

6. The method of claim 1, wherein the second longitudinal side of the second curved laminate defines a right angle at each recess of the plurality of recesses disposed adjacent the periphery of the first longitudinal side, wherein the curved base is adjacent a surface of the embossing roll.

7. The method of claim 1, wherein the recesses are spaced from each other.

8. The method of claim 1, wherein the first curved laminate is disposed at the embossing roll in a laminate holder and wherein the laminate holder extends across a diameter of the embossing roll.

9. The method of claim 1, wherein disposing the first curved laminate at the embossing roll comprises affixing the first curved laminate to the embossing roll.

10. The method of claim 1, wherein causing the material to move into the plurality of cavities comprises engaging a counter pressure roll disposed adjacent the embossing roll to apply a pressure at the material at a portion of the material in contact with a heated portion of the embossing roll thereby advancing the material between the counter pressure roll and the embossing roll.

11. The method of claim 1, wherein the plurality of cavities comprises ten cavities.

12. The method of claim 1, wherein the curved laminates comprise steel.

13. The method of claim 1, wherein the material comprises a homogenous thermoplastic melt.

14. The method of claim 1, wherein the at least a portion of the embossing roll is heated to a temperature above a melting temperature of the material.

15. The method of claim 1, wherein demolding the material from one or more of the first and second curved laminates provides the material having the plurality of projections in a configuration corresponding to a microneedle array.

16. The method of claim 3, wherein a side length of the half-pyramid geometry of at least a recess of the plurality of recesses is 300 micrometer and a base length of the half pyramid geometry of at least a recess of the plurality of recesses is 100 micrometer.

17. The method of claim 3, wherein the half-pyramid geometry of at least a recess of the plurality of recesses corresponds to an aspect ratio between about 1:2 to about 1:4 based on a side length and a base length of the half-pyramid geometry.

18. A microneedle array formed by a process comprising:
disposing at least a first curved laminate adjacent an embossing roll wherein the curved laminate comprises a body having a first longitudinal side and a second longitudinal side extending from a curved base and wherein the first longitudinal side comprises a plurality of recesses disposed adjacent a periphery of the first longitudinal side;
disposing at least a second curved laminate adjacent the embossing roll such that the plurality of recesses of the first curved laminate and a second longitudinal side of the second curved laminate cooperate to define a plurality of cavities there between;
extruding a material onto the at least first and second curved laminates;
heating at least a portion of the embossing roll via an external heat source at a point of contact between the material and one or more of the first and second curved laminates;
causing the material to move into the plurality of cavities to form a plurality of projections at a surface of the material, wherein the projections correspond to the plurality of cavities; and
demolding the material from the at least first and second curved laminates.

19. A system of forming a microneedle array, the system comprising:
a first curved laminate insert adjacent an embossing roll, the first curved laminate insert comprising a body having a first longitudinal side and a second longitudinal side extending from a curved base and wherein the first longitudinal side comprises a plurality of recesses disposed adjacent a periphery of the first longitudinal side; and
at least a second curved laminate insert disposed adjacent the first curved laminate insert such that the plurality of recesses of the first curved laminate insert cooperate with a second longitudinal side of the second curved laminate insert to define a plurality of cavities there between;
a heat source configured to heat at least a portion of the first or second curved laminate insert; and
a counter pressure roll disposed adjacent the first or second curved laminate insert, the counter pressure roll configured to apply a pressure at a material in contact with a heated portion of the first or second curved laminate insert to advance the material between the counter pressure roll and one or more of the first or second curved laminate inserts thereby forming a plurality of projections at a surface of the material, wherein the plurality of projections correspond to a configuration for a microneedle array.

20. The system of claim 19, further comprising a plurality of successive curved laminate inserts forming a plurality of successive cavities configured to receive the material.
